(19) United States
(12) Patent Application Publication
(10) Pub. No.: US 2026/0115479 A1
(43) Pub. Date: Apr. 30, 2026

(54) PACEMAKER CONFIGURATION IDENTIFICATION SYSTEM FOR CARDIAC RESYNCHRONIZATION THERAPY

(71) Applicants: Maurice PIRIO, San Diego, CA (US); The Vektor Group Inc., San Diego, CA (US)

(72) Inventors: Christopher J.T. Villongco, San Diego, CA (US); Robert Joseph Krummen, Bellevue, WA (US); Maurice J. Pirio, Seattle, WA (US)

(21) Appl. No.: 19/479,180

(22) PCT Filed: Apr. 30, 2025
(86) PCT No.: PCT/US2025/027140

§ 371 Date: Oct. 27, 2025

Related U.S. Application Data
(60) Provisional application No. 63/641,844, filed on May 2, 2024.

(51) Int. Cl. A61N 1/362; A61N 1/37; A61N 1/372

(52) U.S. Cl. CPC A61N 1/3627; A61N 1/371; A61N 1/37247

(57) ABSTRACT

A method executed by one or more computing systems involves accessing a cardiogram from a patient, determining an apparent activation location, identifying a pacemaker configuration, creating a visual representation of the patient's heart, marking the apparent activation location on the visual representation, displaying the marked visual representation of the heart, and providing an output indicating the pacemaker configuration.

### CROSS-REFERENCE TO RELATED APPLICATION(S)

This application claims priority to U.S. Provisional Patent Application No. 63/641,844, filed on May 2, 2024, entitled PACEMAKER CONFIGURATION IDENTIFICATION SYSTEM FOR CARDIAC RESYNCHRONIZATION THERAPY, which is hereby incorporated by reference in its entirety.

### BACKGROUND

A pacemaker is device that is designed to manage and maintain the heart's rhythm by delivering electrical pulses to the heart wall. For example, during cardiac resynchronization therapy (CRT), a pacemaker may be employed to treat patients who have an arrhythmia resulting from a complete heart block, also known as a third-degree atrioventricular (AV) nodal block. A complete heart block is a serious condition in which electrical impulses that control the heart's rhythm are blocked from traveling from the atria to the ventricles. A pacemaker may also be employed to treat patients having first-degree heart block, second-degree heart block, bradycardia, atrial fibrillation with slow ventricular response, tachy-brady syndrome, and so on, which may be referred as pacemaker-indicated arrhythmias. A pacemaker has a pulse generator and may have multiple leads which each may have one or more multiple electrodes that can be positioned at various locations on the heart wall. The pulse generator produces electrical pulses that are transmitted to the heart wall via the electrodes to initiate electrical activity in the heart. A pacemaker may also include a sensing mechanism with sensors that can detect intrinsic or extrinsic electrical activity of the heart. A pacemaker may also include a programmable controller for controlling delivery of the electrical pulses and sensing of electrical activity. The programmable controller has various configuration parameters for controlling the delivery of electrical pulses. Based on the parameter settings, the programmable controller controls the timing, output voltage, and output current of the electrical pulses delivered to each electrode. The timing may be at a predefined rate or responsive to detected electrical activity and may factor in a programmable refractory period. The combination of the electrode locations and parameters settings is referred to as the pacemaker configuration.

Many pacemakers have configuration parameters that are based on pacing protocols. Pacing protocols are standardized guidelines that determine how a pacemaker monitors and regulates a patient's heart rhythm. These protocols define the timing (e.g., low rate limit and upper rate limit), location, and pattern of electrical impulses delivered by the device to ensure the heart maintains an adequate rate and rhythm. Pacemakers can be programmed in a variety of pacing modes depending on the patient's specific cardiac condition. Common modes include AAI (atrial pacing and sensing, inhibited response), used in patients with intact AV conduction; VVI (ventricular pacing and sensing, inhibited response), often used in chronic atrial fibrillation; and DDD (dual-chamber pacing and sensing, dual response), which coordinates atrial and ventricular pacing for patients with AV block or sinus node dysfunction. Other specialized modes include DDDR, which adds rate responsiveness to DDD, and VDD, which senses the atrium and paces the ventricle, useful in some cases of AV block with sinus rhythm. CRT devices use modes like VVIR or DDD-BiV to synchronize contractions in patients with heart failure and bundle branch block. Electrophysiologists seek to optimize cardiac output and minimize complications by customizing the parameter settings to each patient's physiology and clinical needs.

When electrical pulses are delivered through multiple electrodes, the electrical activity initiated by the electrical pulses can interact in complex ways and have a resultant effect that is difficult to predict. During a pacemaker implantation procedure, the pacemaker configuration is established (e.g., configuration parameters are set and electrodes placed), and the pacemaker is paced. If the pacing does not have the desired effect, the pacemaker configuration is changed and the pacemaker is paced. This process is repeated until the pacing has the desired effect. The pacemaker can then be implanted based on the last pacemaker configuration. However, the longer the procedure lasts the chance of complications increases, the cost of the procedure increases, and the availability of the electrophysiologist to help other patients decreases.

Even after implantation of a pacemaker, many patients have a suboptimal response to CRT. A patient with a suboptimal response to a CRT is referred to as CRT non-responder, and a patient with a near optimal response to a CRT is referred to as a CRT responder.

It would be desirable to have a technique for pacemaker implantation that would result in shorter implantation procedures by assisting in speeding up the process of identifying an effective pacemaker configuration. In addition, it would be desirable to have a technique that would increase the probability of a patient being a CRT responder.

### BRIEF DESCRIPTION OF THE DRAWINGS

FIG. 1A illustrates an APL for a patient who is a CRT non-responder.

FIG. 1B illustrates an APL for a patient who is a CRT responder.

FIG. 2 illustrates a zone of avoidance for an APL.

FIG. 3 illustrates a target zone for an APL.

FIGS. 4-6 illustrate heart graphics showing different types of information for a target patient.

FIG. 7 illustrates a hotspot indication location of a lead in the left ventricle of the target patient.

FIG. 8 illustrates a hotspot location in the left ventricle after pacing based on the recommendation.

FIG. 9 is a flow diagram that illustrates an example workflow during a pacemaker implantation procedure in some embodiments.

FIG. 10 is a flow diagram that illustrates the processing of a generate simulation library component of the PCI system in some embodiments.

### DETAILED DESCRIPTION

A pacemaker configuration identification (PCI) system is described that provides an indication of an apparent activation location (APL) for a specified pacemaker configuration. The APL represents the location at which activation appears to be initiated even though the electrical pulses are delivered to the electrodes and activation is initiated at each location of an electrode. Knowledge of the APL is helpful in assessing whether the pacemaker configuration would be an effective treatment for a patient. The PCI system may also provide an indication of a pacemaker configuration that would provide a specified APL. The information provided by the PCI system may assist a physician in planning a pacemaker implantation procedure (e.g., identifying a pacemaker configuration) and performing the pacemaker implantation procedure (e.g., based on the identified pacemaker configuration).

If the APL is at a “nonresponsive” location, the heart will not have the desired response. As discussed below, AHA segments 7, 8, 13, 14, and 16 of the 17-segment American Heart Association (AHA) heart model may be associated with a significantly higher probability of poor to suboptimal response to CRT. As a result, these AHA segments are considered to be nonresponsive. An APL within these AHA segments is considered to be at a nonresponsive location. A patient with an APL in one of these AHA segments will likely be a CRT non-responder.

Since the PCI system predicts where the APL will be for a certain pacemaker configuration, the PCI system may be employed to assess the effectiveness of a pacemaker configuration without having to implement and evaluate that pacemaker configuration during an implantation procedure. Before, during, or following a CRT procedure, the PCI system may be employed to determine what the APL would be for a specified pacemaker configuration and/or to determine what pacemaker configuration would result in a specified APL. For example, during an implantation procedure, an electrophysiologist (EP) may implement a certain pacemaker configuration (e.g., standardized pacing protocol), initiate pacing, and evaluate effectiveness of the pacemaker configuration based on analysis of an electrocardiogram (ECG) collected while pacing. If the pacemaker configuration does not have the desired effectiveness or APL, the EP may employ the PCI system to identify a different pacemaker configuration based on an APL specified by the EP. The EP may alter the pacemaker configuration based on the identified pacemaker configuration, initiate pacing, and evaluate the effectiveness of the identified pacemaker configuration. The EP may repeat this process until an effective pacemaker configuration is identified.

In addition, after an implantation procedure, it may be determined that the CRT did not have the desired outcome because, for example, it failed to reverse LV remodeling or improve ejection fraction. The patient may have an anomalous cardiac characteristic that was not known to the EP, and thus, the suggested pacemaker configuration would not have taken cardiac characteristics into consideration. Or the pacemaker configuration was decided upon without the aid of the PCI system. To improve the outcome, the pacemaker configuration may be changed. The PCI system may be employed to, for example, suggest a pacemaker configuration given a new APL provided by an EP. Alternatively, the PCI system may be employed to suggest an alternative pacemaker configuration that may result in the original APL which the current pacemaker configuration was based on. For example, given an APL, the PCI system may suggest multiple pacemaker configurations and assign varying probabilities of generating that APL. As another example, the PCI system may be provided with updated cardiac characteristics and suggest an updated pacemaker configuration or APL factoring in those cardiac characteristics.

Without the PCI system, an EP may repeatedly implement and evaluate the effectiveness of pacemaker configurations that are identified based on the EP's experience. However, because there may be 100s of possible pacemaker configurations (i.e., electrode locations and parameter settings) whose effectiveness depends on many patient-specific cardiac characteristics, an EP may not be able to accurately predict a pacemaker configuration that would be effective. As a result, an EP may implement and evaluate many ineffective pacemaker configurations in search of an effective pacemaker configuration. As discussed in the Background section, such repeated implementations and evaluations tend to prolong procedures that may increase the chances of an adverse procedural outcome, increase the cost of a procedure, and delay procedures for other patients because of the decreased availability of EP labs and EPs.

Rather than employing the PCI system to identify a pacemaker configuration based on a desired APL, the EP may employ the PCI system to evaluate the effectiveness of a specified pacemaker configuration. Prior to or during a procedure, the EP may employ the PCI system to assess the effectiveness of a pacemaker configuration specified by the EP. Given the pacemaker configuration, the PCI system may identify an APL for the pacemaker configuration and/or provide an assessment of its effectiveness. During a procedure, the EP may alter the pacemaker configuration based on their experience and initiate pacing. The PCI system may identify the APL based on a cardiogram collected from the patient while pacing. If the APL is in a nonresponsive AHA segment, the EP may repeat the process until a pacemaker configuration is considered to be effective. The PCI system may also be used to assess a pacemaker configuration prior to or after its implementation to evaluate its effectiveness. A cardiogram may be an electrocardiogram (ECG), a vectorcardiogram (ECG), or more generally any body surface measurement of electrical activity of a heart. Although the PCI system is described primarily with a cardiogram that is an ECG, the PCI system may be used with other body surface measurements.

In some embodiments, the PCI system may provide an assessment of the effectiveness of a specified pacemaker configuration and/or identify an effective pacemaker configuration. The assessment may be based on features derived from the pacemaker configuration, cardiac characteristics of the patient's heart, electrical activity resulting from the pacing of a pacemaker (i.e., resultant electrical activity), a desired APL, a pre-implantation activation location, and so on. The cardiac characteristics may include geometry, scar tissue, conduction velocity, left ventricular ejection fraction, inter-ventricular activation delay, depolarization time, and so on. A pre-implantation activation location may be determined by inputting the pre-implantation cardiogram to a mapping system (described below) which outputs a source location which is considered to be the pre-implantation source location.

In some embodiment, the PCI system generates a two-dimensional, a three-dimensional (3D), or a four-dimensional (4D) graphic of a heart, adds an APL to the graphic, and displays the graphic to help inform a treatment for a patient such as a pacemaker implantation. The PCI system may also display an assessment of the effectiveness of that APL, an indication of a pacemaker configuration that would result in that APL, an indication of a pre-implantation and/or post-implantation cardiogram, and/or a pre-implantation and/or post-implantation activation map. That APL, the pacemaker configuration, the post-implantation cardiogram, and/or the post-implantation activation map may be derived from a simulation of electrical activity (described below) of a heart assuming that APL or the pacemaker configuration. The pre-implantation activation may be generated from a simulation of electrical activity of a heart assuming arrhythmia characteristics but not assuming a pacemaker implantation. The graphic may be generated based on the cardiac characteristics of a simulation or patient-specific cardiac characteristics (e.g., derived from a computed tomography (CT) image). The 4D graphic provides a sequence of 3D graphics derived from the cardiac characteristics at various times within a cardiac cycle. Techniques for generating such a 4D graphic are described in U.S. Pat. No. 12,277,651 titled “3D Cardiac Visualization System” and issued on Apr. 15, 2025, which is hereby incorporated by reference.

The PCI system may employ various machine learning (ML) models and non-ML algorithms to assist in the identification of a pacemaker configuration that would be effective at treating an arrhythmia of a patient. The ML models and the non-ML algorithms may be combined in various ways, for example, to identify a pacemaker configuration or an APL that may be effective at treating an arrhythmia.

The PCI system may employ (1) an evaluate APL ML model that evaluates the effectiveness of a designated APL, (2) a predict APL ML model that predicts what the APL would be for a designated pacemaker configuration, (3) a predict PC ML model that predicts what pacemaker configuration would result in a designated APL, (4) a suggest APL ML model that suggests an APL for treating the patient, and (5) a suggest PC ML model that suggests a pacemaker configuration for treating a patient.

By employing the ML models, the PCI system overcomes disadvantages of techniques that do not employ machine learning. For example, one such technique may identify an APL by searching a library of mappings of pacemaker configurations and cardiac characteristics to APLs. The searching may involve comparing a proposed pacemaker configuration and a patient's cardiac characteristics to millions of mappings to identify mappings with similar pacemaker configurations and cardiac characteristics. However, hundreds of the mappings may be considered similar. For example, one mapping may be consider similar primarily because the pacemaker configurations are very similar but the cardiac characteristics are not so similar. In contrast, another mapping may be considered similar because both the cardiac characteristics and the pacemaker configurations are somewhat similar, but neither is very similar. When many mappings are identified, the process of manually identifying an APL from those mappings can be time consuming and subjective resulting in patient outcomes that may be less than optimal. In addition, because the searching can be computationally expensive and very time consuming, it may not be practical to identify an APL in real time during an implantation procedure. In such as case, the identification would not be based on cardiac characteristics that are identified during the implantation procedure.

An ML model that predicts an APL based a pacemaker configuration and cardiac characteristics overcomes these disadvantages. First, the identification can be done in real time during an implantation procedure because applying weights of the ML model is neither computationally expensive nor time consuming. Although the training of the ML model can be both computationally expensive and time consuming, the training needs only be performed once and the learned weights can be applied any number of times. Second, the training of the ML model learns how pacemaker configurations and cardiac characteristics influence an APL based on training examples (i.e., mappings). Thus, the applying of the weights of the ML model can identify accurate APLs even though the training examples may not have pacemaker configurations and cardiac characteristics that are similar to those to which the ML model is applied. Each of the ML models of the PCI system has similar advantages.

The evaluate APL ML model inputs a feature vector that includes features derived from an APL and features derived from patient cardiac characteristics and/or a patient ECG and outputs an indication of the effectiveness of that APL in treating the patient. The indication may be a probability that the patient would be a CRT responder.

The predict APL ML model inputs a feature vector with features derived from a pacemaker configuration and features derived from patient cardiac characteristics and/or a patient ECG and outputs an indication of a predicted APL that may result from that pacemaker configuration for the patient. The predict PC ML model inputs a feature vector with features derived from an APL and features derived from patient cardiac characteristics and/or a patient ECG and outputs an indication of a pacemaker configuration that may result in that APL for the patient.

The suggest APL ML model inputs a feature vector with features derived from patient cardiac characteristics and/or a patient ECG and outputs a suggested APL for treating the patient. The suggest PC ML model inputs features derived from patient cardiac characteristics and/or a patient ECG and outputs an indication of a pacemaker configuration that may be successful in treating the patient. A feature of an ECG may be an ECG voltage-time series, a QRS integral, S-T segment time, and so on.

In some embodiments, rather than (or in addition to) inputting a cardiogram, an ML model or a non-ML algorithm may input a pre-implantation activation location. Such a pre-implantation activation location may be considered to be derived from a cardiogram such as when identified by a mapping system. In some embodiments, an ML model or a non-ML algorithm may input values a subset of the parameter settings and/or a subset of electrode placements. The subsets may be proper, improper, and empty subsets. For example, all the electrode placements may be input to an ML model or a non-ML algorithm which outputs values for parameter settings of a pacemaker. As another example, values for a proper subset of the parameter settings may be input to an ML model or a non-ML model which outputs electrode placements and values for the other parameter settings.

In some embodiments, multiple ML models may be combined into integrated ML models. For example, the predict APL ML model (or the suggest APL ML model) and the evaluate APL ML model may be integrated into an ML model that outputs both a predicted APL and an indication of its effectiveness. As another example, the suggest APL model and the predict PC ML model may be integrated into an ML model that inputs a feature vector with features derived from patient cardiac characteristics and/or a patient ECG and outputs both a suggested APL and a predicted pacemaker configuration that would provide the suggested APL. The ML models may also be employed sequentially. For example, the suggest APL ML model may be employed to determine a suggested APL, and the predict PC ML model may input the suggested APL and output a predicted pacemaker configuration that would have the suggested APL.

The ML models may be trained using a PC library of records that each has fields that store a pacemaker configuration, an APL, an ECG, an indication of effectiveness, cardiac characteristics, and so on. The PC library may be a clinical library, a simulation library, or a combination of them. A clinical library is derived from electronic health records (EHRs) of patients who have had a pacemaker implantation. The PCI system also employ a mapping system (described below) to determine the APLs of the clinical library. For each EHR, the PCI system inputs the patient ECG into the mapping system which outputs a source location that is considered to be an APL. A simulation library may be derived from simulations of electrical activity of a heart that are each based on a pacemaker configuration and cardiac characteristics. The PCI system may identify a simulated APL given a simulated ECG that is generated from a simulation of electrical activity. Various techniques may be employed to analyze an ECG to determine whether the APL would be result in a desired cardiac rhythm (i.e., would be effective). Techniques for assessing cardiac rhythm are described in Surawicz, B., Childers, R., Deal, B. J. and Gettes, L. S., 2009. AHA/ACCF/HRS recommendations for the standardization and interpretation of the electrocardiogram: part III: intraventricular conduction disturbances: a scientific statement from the American Heart Association Electrocardiogramd Arrhythmias Committee, Council on Clinical Cardiology; the American College of Cardiology Foundation; and the Heart Rhythm Society: endorsed by the International Society for Computerized Electrocardiology. Circulation, 119 (10), pp. e235-e240, which is hereby incorporated by reference.

Rather than employing an ML model, the PCI system may employ a non-ML algorithm to identify a pacemaker configuration and/or an APL and associated effectiveness. The PCI system may search a PCI library given the cardiac characteristics and/or an ECG of a patient to identify a pacemaker configuration that may be effective in treating the patient. The PCI system may use the cardiac characteristics (e.g., cardiac geometry) of the patient to identify records associated with similar cardiac characteristics. From the identified records, the PCI system identifies the records of CRT responders and analyzes their pacemaker configurations. If many of the pacemaker configurations (e.g., above a threshold percentage) are very similar, the PCI system may indicate that those pacemaker configurations may be effective. Similarly, to identify what the APL would be for a specified pacemaker configuration, the PCI system may search the identified records for a pacemaker configuration that is similar to the specified pacemaker configuration. The PCI system then assesses whether APLs of the identified records are similar (e.g., based on a similarity criterion such as within a specified distance that may be 2 mm). If the APLs are similar, the PCI system provides an indication of those APLs. If the APLs are not similar, the PCI system may indicate that APL cannot be identified from the data of the PCI library.

To generate the simulation library, the PCI system runs simulations of electrical activity of a heart that are based on pacemaker configurations, cardiac characteristics, arrhythmia origin (which may be considered a cardiac characteristic), and so on. The PCI system generates computational models that are each based on cardiac characteristics and an arrhythmia origin. For each computational model, the PCI system runs a simulation of electrical activity, assuming a pacemaker configuration or APL, to simulate the electrical activity of the heart at simulation intervals (e.g., 1 ms). Each simulation is run until the simulated electrical activity stabilizes (e.g., into normal sinus rhythm), for a predefined time period, and so on. Each simulation is based on a three-dimensional (3D) mesh representing a cardiac geometry. A 3D mesh is a finite-element mesh that has vertices that correspond to locations within the heart wall (endocardium, myocardium, and epicardium) and that are associated with cardiac characteristics such as fiber orientation and conduction velocity. Each vertex is also associated with the simulated electrical activity of each simulation interval. For example, the left ventricle may be defined as having approximately 70,000 heart locations with the 3D mesh storing an electromagnetic value for each heart location. If so, a three-second simulation with a step size of 1 ms would generate 3,000 electromagnetic values for each of the 70,000 vertices. Simulations of electrical activity of a heart are described in Villongco, C. T., 2015. Patient-specific Computational Models of Dyssynchronous Heart Failure and Cardiac Resynchronization Therapy for Clinical Diagnosis and Decision Support. University of California, San Diego, described in Villongco, C. T., Krummen, D. E., Omens, J. H. and McCulloch, A. D., 2016. Non-invasive, model-based measures of ventricular electrical dyssynchrony for predicting CRT outcomes. EP Europace, 18 (suppl_4), pp. iv104-iv112, and described in Crozier, A., Blazevic, B., Lamata, P., Plank, G., Ginks, M., Duckett, S., Sohal, M., Shetty, A., Rinaldi, C. A., Razavi, R. and Niederer, S. A., 2016. Analysis of lead placement optimization metrics in cardiac resynchronization therapy with computational modelling. EP Europace, 18 (suppl_4), pp. iv113-iv120, which are hereby incorporated by reference. The PCI system may also generate a simulated ECG for each simulation based on the simulated electrical activity of that simulation. If the simulated ECG has characteristics that indicate the pacemaker configuration or APL of the simulation would be effective at treating a pacemaker-indicated arrhythmia, the simulation may be indicated as a CRT responder.

For each simulation, the PCI system adds a record to the simulation library. A record may include the computational model, pacemaker configuration of the simulation, a simulated cardiogram (ECG or vectorcardiogram (VCG)), an APL, effectiveness score, and so on. The simulated cardiogram is generated based on the electrical activity associated with the vertices and assuming a thorax configuration (e.g., size, fat distribution, and so on), which may be the same for each simulation. The APL may be identified using a mapping system that inputs the cardiogram and outputs an APL. The mapping system may employ an ML model or a non-ML algorithm. Mapping systems are described in U.S. Pat. No. 11,380,055 titled “Computational Localization of Fibrillation Sources” and issued on Jul. 5, 2022 and U.S. Pat. No. 10,860,754 titled “Calibration of Simulated Cardiograms” and issued on Dec. 8, 2020, which are hereby incorporated by refence. An APL may be represented as a probability distribution (e.g., Gaussian) with an x-axis representing locations and y-axis representing probabilities for the locations. The highest probability may be associated with the most likely APL. The effectiveness score may be based on confidence in the accuracy of the APL, closeness of the APL to a nonresponsive segment, and so on. The effectiveness score may also be based on dyssynchrony that is determined from analysis of the simulated cardiogram. For example, a wide QRS complex (e.g., >120 ms) and variations in QRS morphology across the 12 ECG leads may indicate a delayed left ventricular depolarization.

In some embodiments, prior to running the simulations based on a pacemaker configuration or an APL, the PCI system may first run arrhythmia simulations of electrical activity to simulate electrical activity associated with various pacemaker-indicated arrhythmias and generate a simulated arrhythmia ECG for each simulation. Each arrhythmia simulation may run until the electrical activity stabilizes into an arrhythmia. For each such arrhythmia simulation, the PCI system may run various continuations of that arrhythmia simulation assuming various pacemaker configurations or APLs, generate a simulated ECG for each simulation, and assess whether the pacemaker configuration or APL may be effective at treating the arrhythmia. Given a patient ECG, the PCI system may identify a matching simulated arrhythmia ECG and then use the continuations of the simulation from which the matching simulated arrhythmia ECG was generated to determine a pacemaker configuration or APL for the patient. Techniques for determining stability are described Krummen, D., et al., Rotor Stability Separates Sustained Ventricular Fibrillation from Self-Terminating Episodes in Humans, Journal of the American College of Cardiology, Vol. 63, No. 24, 2014, which is hereby incorporated by reference.

In some embodiments, the PCI system may run patient-specific simulations that are based on cardiac characteristics of a patient. The cardiac characteristics may be derived from images (e.g., computed tomography imaging) of the patient's heart. From the images, the PCI system generates a 3D mesh based on the patient's cardiac characteristics. Techniques for generating such a 3D mesh are described in Int'l. Pub. No. WO 2023/168017 titled “Overall Ablation Workflow System” and published on Sep. 7, 2023, which is hereby incorporated by reference. The PCI system may determine the source location of the patient's arrhythmia using a mapping system. The PCI system then runs various simulations that each assume a pacemaker configuration or an APL. If a simulation does not result in an arrhythmia, the pacemaker configuration or the APL may be considered to be effective at treating the patient. Each patient-specific simulation may start with a 3D mesh representing the electrical activity based on a patient-specific arrhythmia simulation or a non-patient specific arrhythmia simulation. The various simulations that are described herein may be bootstrapped based on a prior simulation. Techniques for bootstrapping a simulation are described in the '754 patent. In addition, the selection of a simulation, for example, to identify a pacemaker configuration, an APL, or a source location may be from simulations based on cardiac characteristics that are similar to the patient's cardiac characteristics. Techniques for such selection are described in the '754 patent.

In some embodiments, the PCI system identifies AHA segments that are nonresponsive based on analysis of clinical data of patients who are CRT non-responders and patients who are CRT responders. For each patient, the PCI system may input the ECG of that patient collected after CRT to the mapping system which outputs an APL. The PCI system then identifies the AHA segment that contains that APL, may associate the APL with the AHA segment, and may increment a count of CRT non-responders or count of CRT responders for that AHA segment based on whether the patient is a CRT non-responder or CRT responder. After processing all the clinical data, the PCI system analyzes the counts and/or APLs associated with the AHA segments to determine which AHA segments are nonresponsive. For example, if the number of CRT non-responders for an AHA segment is greater that an “nonresponsive threshold percentage” (e.g., 95%), the PCI system designates that AHA segment as nonresponsive. As discussed above, an analysis of clinical data has indicated that AHA segments 7, 8, 13, 14, and 16 are nonresponsive. To provide assessment that is more granular than an AHA segment, the PCI system may generate a probability distribution of non-responsiveness for each AHA segment. For example, the probability may be higher near the middle part of the AHA segment and lower near an outer part which may be near an AHA segment that is designated as responsive. When determining whether an APL is responsive, the PCI system may provide a probability based on the probability distribution associated with the AHA segment that contains the APL. The probability may help an EP in assessing the effectiveness of a pacemaker configuration.

In some embodiments, the PCI system may provide separate implementations for different models of pacemakers that may have different leads and electrodes and/or different configurable parameters. For example, one model may have a sensor, but another model does not. As another example, one model may have a lead with 8 electrodes and another model may have a lead with 4 electrodes.

The term “similarity” is used in conjunction with various types of data such as an APL, a pacemaker configuration, and an ECG. Similarity may be represented by a similarity score. Also, data is considered to be similar when the data satisfies a “similarity criterion” such as the similarity score being greater than a similarity score threshold. As an example, the similarity for APLs may be based on distance between the APL (e.g., <3 mm). The similarity for pacemaker configurations may be based on a vector with elements corresponding to parameters and lead locations. The similarity may be determined using, for example, cosine similarity or Pearson correlation. The similarity for ECGs may be based on features derived from the ECGs (e.g., QRS integral and width and length of the S-T interval) or based on a distance derived from the voltage-time series of the ECG using, for example, cosine similarity or Pearson correlation.

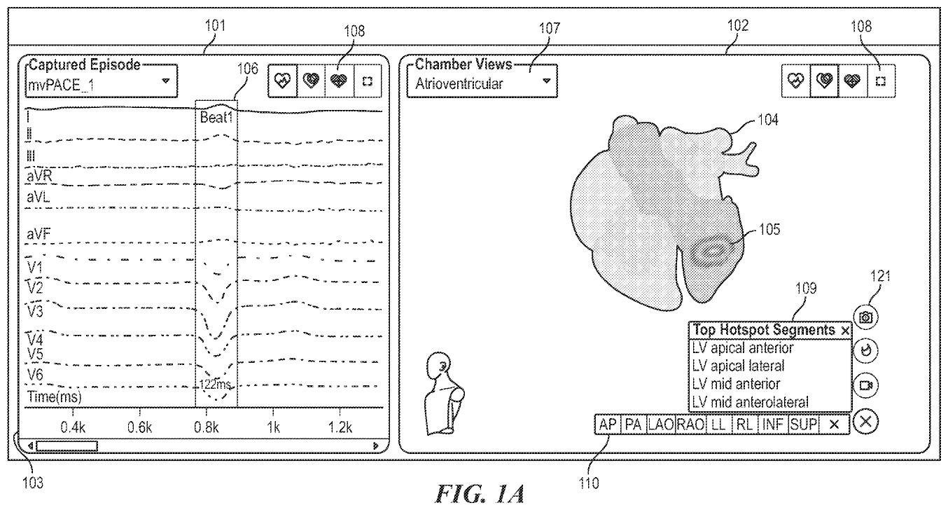

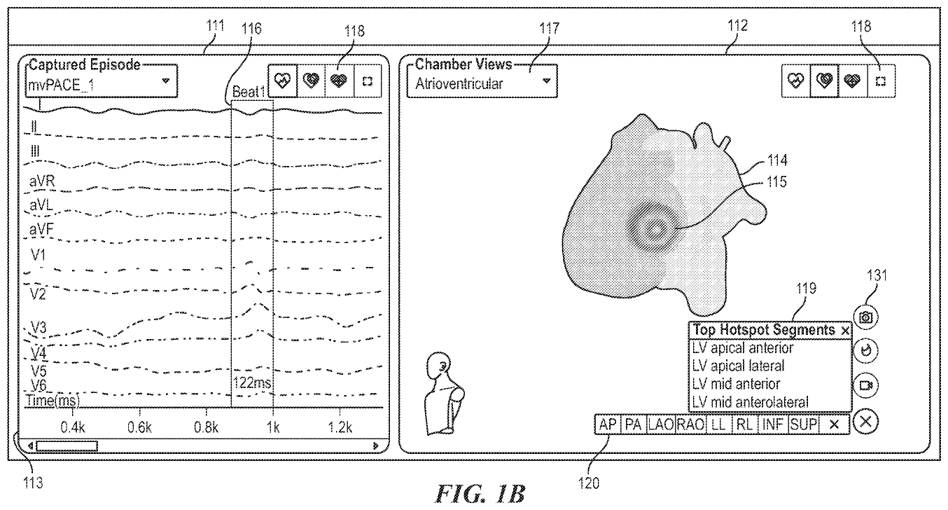

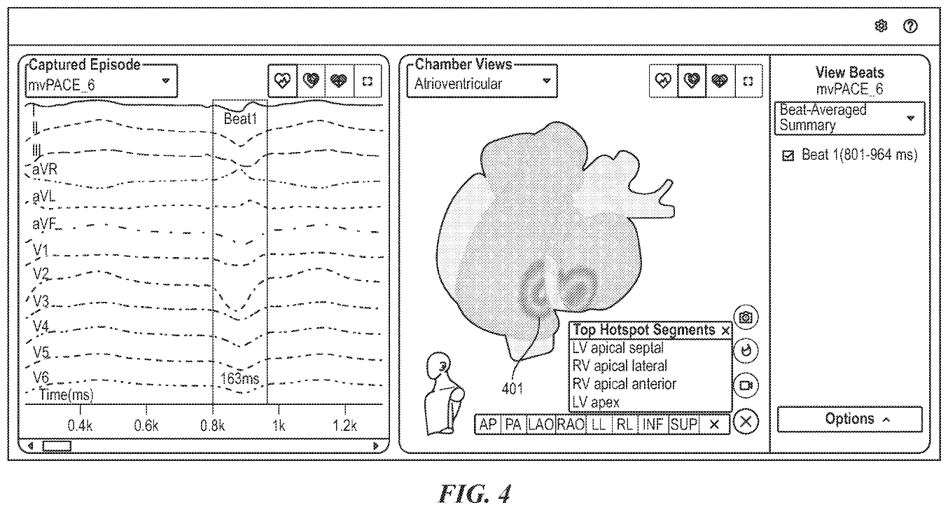

FIGS. 1-8 illustrate a user interface of the PCI system in some embodiments. Each of FIGS. 1-3 include a left panel and a right panel, and FIGS. 4-8 include a left panel, a middle panel, and a right panel. In FIGS. 1-8, the APL is referred to as the “summed biventricular activation source location.”

FIG. 1A illustrates an APL for a patient who is a CRT non-responder. FIG. 1A, left panel 101, shows an ECG 103 collected during biventricular pacing of the patient. FIG. 1A, right panel 102, shows a heart graphic 104 of the heart of the patient. The APL 105 is shown by the shaded area, referred to as a hotspot, on the heart graphic. The hotspot indicates that the APL is in the apical anterior portion of the left ventricle. The center of hotspot is white, indicating the region with the highest probability of containing the APL. The circular shading surrounding the white region indicate regions of decreasing probability of containing the APL (e.g., based on a probability distribution) with the closest surrounding region surrounding having the second highest probability. The white region (in segment 13) is highly correlated with lack of response to CRT (e.g., lack of improvement in left ventricular ejection fraction, lack of clinical improvement in heart failure symptoms, lack of reduction in left ventricular end-diastolic dimension, etc.). In this example, the patient is a CRT non-responder because the patient did not experience a significant improvement in left ventricular ejection fraction after implantation of the cardiac resynchronization device.

FIG. 1B illustrates an APL for a patient who is a CRT responder. FIG. 1B, left panel 111, shows an ECG 113 collected during biventricular pacing for the patient. FIG. 1B, right panel 112, shows a heart graphic 114 of the heart of the patient. The APL 115 is indicated by a hotspot in the mid, inferolateral left ventricle. The white region (in segment 11) is highly correlated with a positive response to CRT (e.g., improvement in left ventricular ejection fraction, improvement in heart failure symptoms, reduction in left ventricular end-diastolic dimension, etc.). In this example, the patient is a CRT responder because the patient experienced a significant improvement in left ventricular ejection fraction and in heart failure symptoms after implantation of the cardiac resynchronization device.

The PCI system allows a user to select an ECG portion of the ECG to be used in determining the APL. In FIGS. 1A and 1B, the ECG portions 106 and 116 are each illustrated by the shaded vertical rectangle that extends from lead I through lead V6. The ECG portion of FIG. 1A spans approximately 780 ms to 880 ms of the cardiac cycle. In some embodiments, rather than using the user-selected ECG portion, the PCI system may automatically select the ECG portion or generate a refined ECG portion from the selected ECG portion. Use of a refined ECG portion may more likely result in the identification of a more accurate APL. Techniques for generating a refined ECG portion are described in PCT Pub. No. 2024/044719 titled “Automatic Refinement of Electrogram Selection” and published on Feb. 29, 2024, which is hereby incorporated by reference. The PCI system allows a user to scroll through an ECG to display and allow selection of one or more ECG portions from different cardiac cycles.

The PCI system allows a user to rotate the graphic of the heart through any orientation, that is, any spherical angle (e.g., azimuthal angle and polar angle.) By rotating the graphic, the user may better understand the spatial placement of electrodes, the APL, and the nonresponsive segments. Although not illustrated, the PCI system may also display an indication of the placement of the leads including electrodes which may be label with lead/electrode identifiers. The graphic of the heart may be generated from a 3D image (e.g., CT scan) of the patient's heart. The PCI system may employ the Blender open-source system to generate, display, and rotate the graphic. (Blender Foundation, Blender, version 2.93, Blender, 2023. [Online]. Available: https://www.blender.org/.) Various techniques for generating a 3D graphic and for augmenting the 3D graphic with a source location that is an origin of electrical activation are described in PCT Pub. No. 2023/168017 titled “Overall Ablation Workflow System” and published on Sep. 7, 2023, which is hereby incorporated by reference.

The PCI system allows a user to select the chambers that the heart graphic represents. In FIGS. 1A and 1B, the user has selected the atrioventricular view from the drop-down lists 107 and 117 (upper left corner of right panel). The upper right corners of the left panel and the right panel provide icons 108 and 118 for selecting the information to display in a panel including an ECG, a heart graphic, and an intra-cardiogram similarity graphic. An intra-cardiogram similarity graphic provides a grid that illustrates the similarity between cardiac cycles or portions of cardiac cycles of an ECG. The horizonal and vertical axes of the grid identify the cycles and the intersection of a cycle of the horizontal axis and a cycle of the vertical axis represents the similarity between the cycles (e.g., using colors). The intersections along the 45-degree line have a similarity that indicates the cycles are identical because each intersection is between the same cycle on the horizontal axis and the vertical axis. An intra-cardiogram similarity graphic is described in U.S. Pat. No. 10,595,736 titled “Heart Graphic Display System” and issued on Mar. 24, 2020, which is hereby incorporated by reference.

FIGS. 1A and 1B also list the hotspot segments (e.g., AHA segments) in the windows 109 and 119 at the lower right portion of the right panel. The hotspot segments are the LV apical anterior (segment 13), the LV apical lateral (segment 16), the LV mid anterior (segment 7), and the LV mid anterolateral (segment 11). The horizontal areas 110 and 120 at the bottom of the right panel indicates that various “standard views” can be selected such as “AP” for anterior-posterior and “INF” for inferior. In addition, a user can change the view manually by touching the display and moving their finger in a direction to indicate the rotation. The icons 121 and 131 at the lower right corners include a camera icon, a hotspot display icon, a video icon, and an X icon. The camera icon is selected to take a snapshot of the heart graphic. The hotspot display icon (second icon) is selected to display the hotspot segments. The video icon and the X icon are selected to open and close the blue horizontal area.

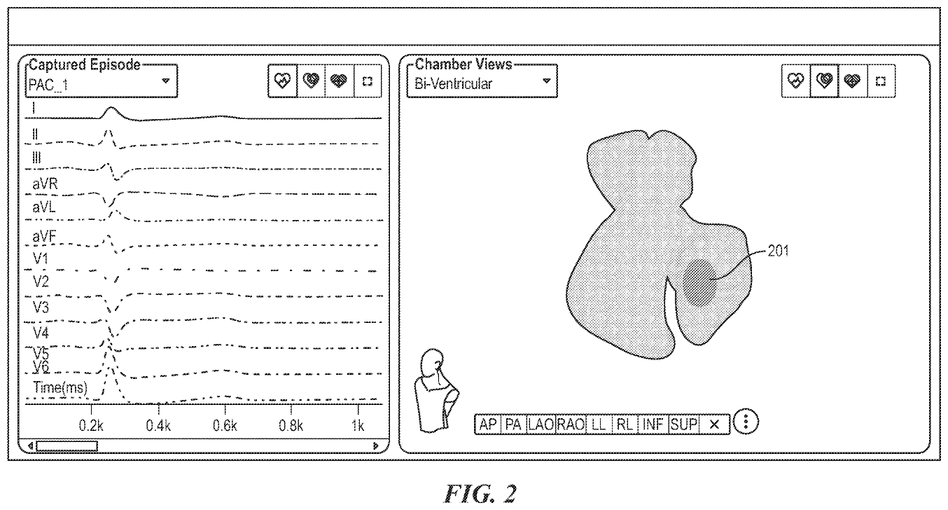

FIG. 2 illustrates a zone of avoidance for an APL. The zone of avoidance 201 corresponds to a nonresponsive segment(s). The right panel shows a left anterior oblique view of the heart with the zone of avoidance indicated by the shaded area. A zone of avoidance may correspond to one or more AHA segments that are nonresponsive.

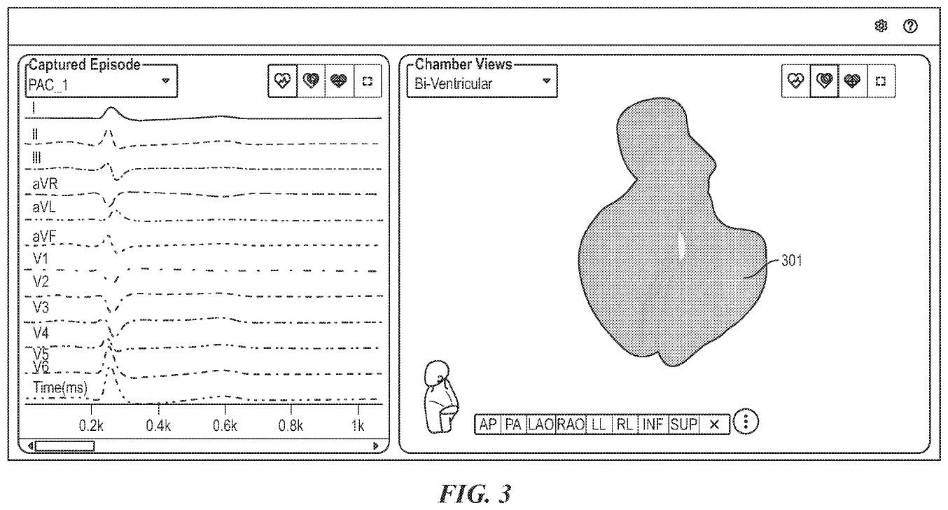

FIG. 3 illustrates a target zone for an APL. The right panel shows a left anterior oblique cranial view of the heart with the target zone 301 indicated by the shaded area. The right panel may also show intermediate zones (also referred to acceptable zones) within the left or right ventricles. If an APL is in an intermediate zone, the patient may be responsive to CRT. A zone of avoidance, a target zone, and an intermediate zone may be indicated in by level of shading. The shading of zones may indicate probability of the patient being a CRT responder. Rather than using shadings in the figures, coloring may be employed with, for example, green representing a high probability of responding and red representing a low probability of responding.

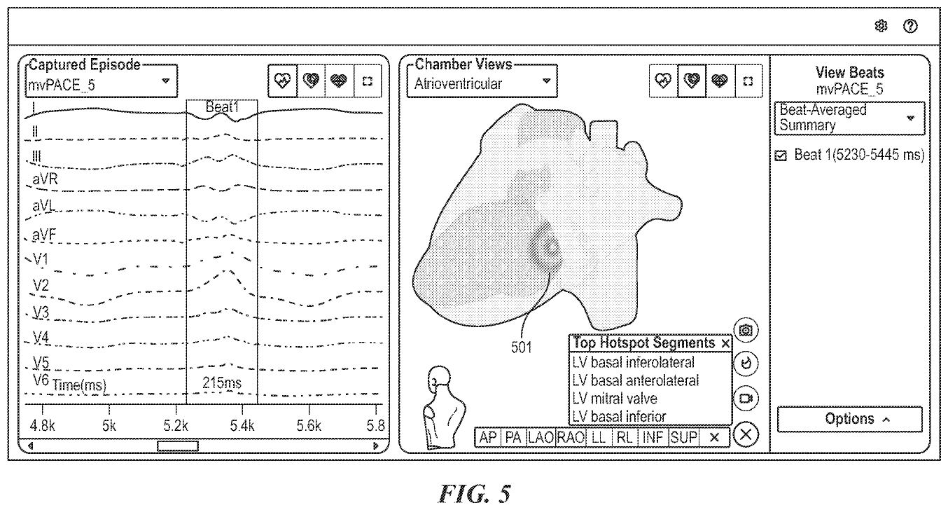

FIGS. 4-6 illustrate heart graphics showing different types of information for a target patient. FIGS. 4 and 5 each illustrate a single lead position. In FIG. 4, the middle panel shows a calculated lead position for a lead in the right ventricle. The calculated lead position is indicated by the hotspot 401 in the right ventricle. The coloring indicates probability of the lead position in a manner similar to a hotspot showing an APL. In FIG. 5, the middle panel shows a calculated lead position for a lead in the left ventricle. The calculated lead position is indicated by the hotspot 501 in the left ventricle.

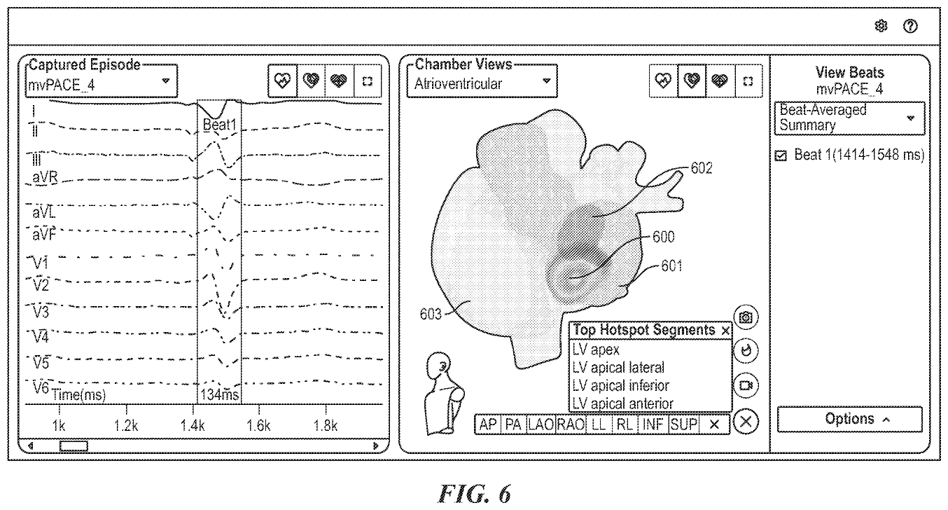

FIG. 6 illustrates a hotspot of an APL, a zone of avoidance, a target zone, and an intermediate zone. The APL is indicated by the hotspot 600 at the apex of the left ventricle. The target zone 601 includes segments 9, 10, 11, 12, 15, and 16, the zone of avoidance 602 includes segments 7, 8, 13, 14, and 16, and the intermediate zone is in the right ventricles. In this example, the hotspot for the APL is within segment 17 which is consistent with electrical activations resulting from electrical pulses delivered by leads positioned as illustrated by FIGS. 4 and 5.

The right panel of FIGS. 4-6 indicates the cardiac cycles (beat) used in identifying the hotspots. When multiple beats are used, the average of the beats is used to identify the hotspots. The cardiac cycles are indicated in the right panels.

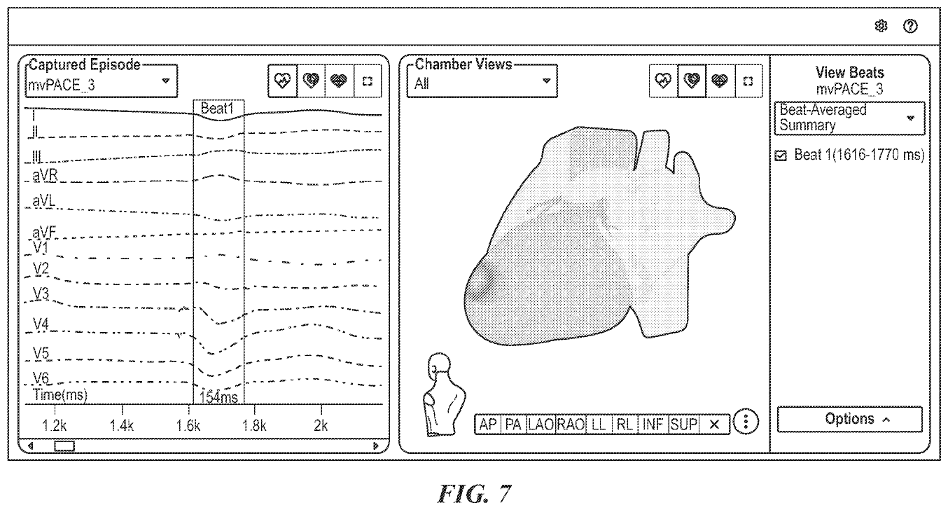

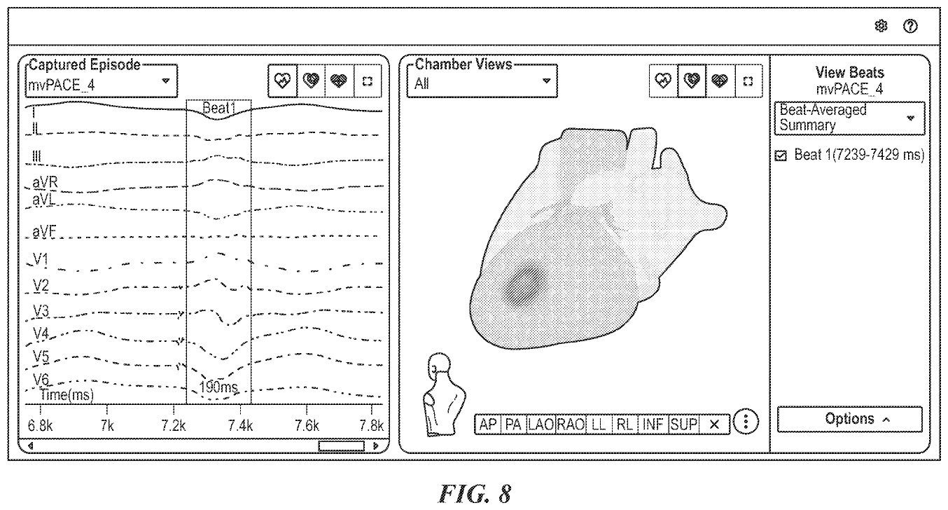

FIG. 7 illustrates a hotspot indication location of a lead in the left ventricle of the target patient. The lead is paced with electrode 1 being the cathode and electrode 2 being the anode represented as 1→2 (cathode→anode designation). The pacing of this lead indicates a location in the anteroapical portion of the heart (i.e., segment 13). Such a location increases the probability of the APL being in the zone of avoidance (not shown). The PCI system may be employed to recommend changes to the pacemaker configuration (e.g., lead locations or parameter settings). The recommendations may be to pace electrodes 4→3. FIG. 8 illustrates a hotspot location in the left ventricle after pacing based on the recommendation. The pacing of the lead indicates a location in the mid anterolateral region of the left ventricle (i.e., segment 12). Such a location increases the probability of the APL being in the target zone (not shown) which increases the probability of the patient being a CRT responder.

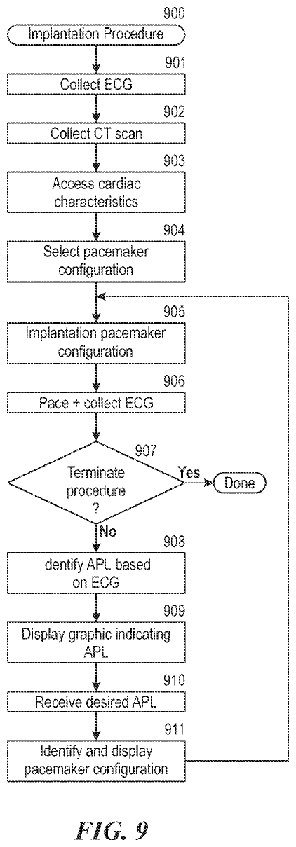

FIG. 9 is a flow diagram that illustrates an example workflow during a pacemaker implantation procedure in some embodiments. The implantation procedure component 900 provides information to an EP during an implantation procedure to help inform a successful procedure and overall successful CRT. In block 901, the component collects an ECG from the patient. In block 902, the component collects a CT scan of the heart of the patient. In block 903, the component accesses cardiac characteristics of the patient. The cardiac characteristics may be based on the EHR of the patient and/or the CT scan. In block 904, the component selects a pacemaker configuration that may be specified by the EP. generated by the PCI system based on an APL provided by the EP, and so on. In block 905, the component receives an indication that the pacemaker configuration has been implemented. In some embodiments the PCI system may interface directly with the pacemaker to establish the pacemaker configuration or provides the pacemaker configuration to a computing system that interfaces with the pacemaker to establish the pacemaker configuration. In block 906, the component directs the pacing and collecting of an ECG. In decision block 907, if the EP indicates that the procedure should be terminated, then the component completes, else the component continues at block 908. The EP may make the decision to terminate based on analysis of the ECG. Alternatively, the component may perform the processing of blocks 908 and 909 to provide more information to the EP that may be used to determine whether to terminate the procedure. In block 908, the component identifies an APL based on the ECG using the PCI system. In block 909, the component displays a graphic of the patient's heart derived from the ECG that indicates the APL. The graphic may indicate other information such as zones of avoidance, the actual target zone, and intermediate zones. In block 910, the component receives a desired APL from the EP. In block 911, the component identifies and displays information relating to the pacemaker configuration that may result in the desired APL. The pacemaker configuration may be identified using the PCI system based on the desired APL. The pacemaker configuration information may be displayed as a list of parameters and corresponding parameter values along with a graphic that indicates the locations of the electrodes. The component then loops to block 905 to implement the pacemaker configuration.

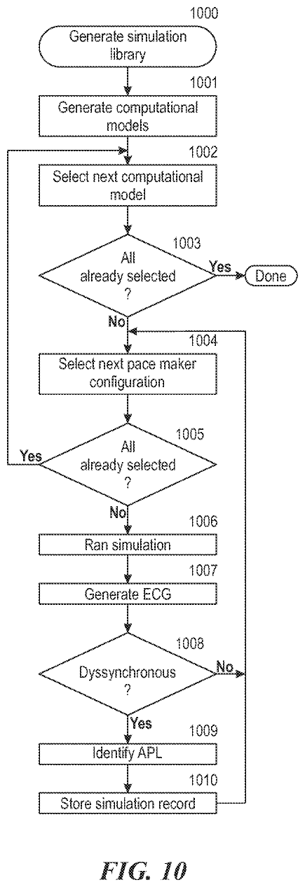

FIG. 10 is a flow diagram that illustrates the processing of a generate simulation library component of the PCI system in some embodiments. The generate simulation library component 1000 is invoked to run simulations and create the simulation library based on those simulations. In block 1001, the component generates computational models to be used in the simulations. The computational models represent various cardiac characteristics such as cardiac geometries, electrical characteristics (e.g., conduction velocities), scar tissue, fiber architectures, and so on. In block 1002, the component selects the next computational model. In decision block 1003, if all the computational models have already been selected, then the component completes, else the component continues at block 1004. In block 1004, the component selects the next pacemaker configuration out of a collection of possible pacemaker configurations. In decision block 1005, if all the pacemaker configurations have already been selected, then the component loops to block 1002 to select the next computational model, else the component continues at block 1006. In block 1006, the component runs a simulation based on the selected computational model and the selected pacemaker configuration. In block 1007, the component generates an ECG based on the simulation. In decision block 1008, if the ECG indicates that the cardiac activity is dyssynchronous, then the component continues at block 1009, else the component loops to block 1004 to select the next pacemaker configuration. In block 1009, the component identifies an APL based on the ECG using a mapping system. In block 1010, the component stores in the simulation library a simulation record that includes the computational model, the pacemaker configuration, the ECG, and the APL. The component then loops to block 1004 to select the next pacemaker configuration.

The computing systems (e.g., network nodes or collections of network nodes) on which the PCI system and the other described systems may be implemented may include a central processing unit, input devices, output devices (e.g., display devices and speakers), storage devices (e.g., memory and disk drives), network interfaces, graphics processing units, communications links (e.g., Ethernet, Wi-Fi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, distributed systems, cloud-based computing systems, client computing systems that interact with cloud-based computing system, desktop computers, laptops, tablets, e-readers, personal digital assistants, smartphones, gaming devices, servers, and so on. The computing systems may access computer-readable media that include computer-readable storage mediums and data transmission mediums. The computer-readable storage mediums are tangible storage means that do not include a transitory, propagating signal. Examples of computer-readable storage mediums include memory such as primary memory, cache memory, and secondary memory (e.g., DVD), and other storage. The computer-readable storage media may have recorded on them or may be encoded with computer-executable instructions or logic that implements the PCI system and the other described systems. The data transmission media are used for transmitting data via transitory, propagating signals or carrier waves (e.g., electromagnetism) via a wired or wireless connection. The computing systems may include a secure crypto processor as part of a central processing unit (e.g., Intel Secure Guard Extension (SGX)) for generating and securely storing keys and for encrypting and decrypting data using the keys and for securely executing all or some of the computer-executable instructions of the PCI system. Some of the data sent by and received by the PCI system may be encrypted, for example, to preserve patient privacy (e.g., to comply with government regulations such the European General Data Protection Regulation (GDPR) or the Health Insurance Portability and Accountability Act (HIPAA) of the United States). The PCI system may employ asymmetric encryption (e.g., using private and public keys of the Rivest-Shamir-Adleman (RSA) standard) or symmetric encryption (e.g., using a symmetric key of the Advanced Encryption Standard (AES)).

The one or more computing systems may include client-side computing systems and cloud-based computing systems (e.g., public or private) that each executes computer-executable instructions of the PCI system. A client-side computing system may send data to and receive data from one or more servers of the cloud-based computing systems of one or more cloud data centers. For example, a client-side computing system may send a request to a cloud-based computing system to perform tasks such as run a identify a pacemaker configuration given an APL or identify a pacemaker configuration given an ECG. A cloud-based computing system may respond to the request by sending to the client-side computing system data derived from performing the task such as a pacemaker configuration or APL. The servers may perform computationally expensive tasks in advance of processing by a client-side computing system such as training an ML model or in response to data received from a client-side computing system. A client-side computing system may provide a user experience (e.g., user interface illustrated in FIGS. 1-8) to a user of the PCI system. The user experience may originate from a client computing device or a server computing device. For example, a client computing device may generate a patient-specific graphic of a heart and display the graphic. Alternatively, a cloud-based computing system may generate the graphic (e.g., in a Hyper-Text Markup Language (HTML) format, an eXtensible Markup Language (XML) format or a Digital Imaging and Communications in Medicine (DICOM) format) and provide it to the client-side computing system for display. A client-side computing system may also send data to and receive data from various medical devices such as an ECG monitor, a pacemaker, a CRT planning device, and so on. The data received from the medical devices may include an ECG, cardiac characteristics of a patient, CRT scan, and so on. The data sent to a medical device may include data, for example, in the DICOM format. A client-side computing device may also send data to and receive data from medical computing systems. Such medical computing systems store patient medical history (EHRs) data, descriptions of medical devices (e.g., type, manufacturer, and model number) of a medical facility, medical facility device descriptions, results of procedures, and so on. The term cloud-based computing system may encompass computing systems of a public cloud data center provided by a cloud provider (e.g., Azure provided by Microsoft Corporation) or computing systems of a private server farm (e.g., operated by the provider of the PCI system).

The PCI system and the other described systems may be described in the generated text of computer-executable instructions, such as program modules and components, executed by one or more computers, processors, or other devices. Generally, program modules or components include routines, programs, objects, data structures, and so on that perform tasks or implement data types of the PCI system and the other described systems. Typically, the functionality of the program modules may be combined or distributed as desired in various examples. Aspects of the PCI system and the other described systems may be implemented in hardware using, for example, an application-specific integrated circuit (ASIC), graphic processing units (GPU), or a field programmable gate array (FPGA). For example, a GPU may be employed when training an ML model.

An ML model employed by the PCI system may be any of a variety or combination of supervised, semi-supervised, self-supervised, unsupervised, or reinforcement learning ML models including a neural network (NN) such as fully connected, convolutional, recurrent, or autoencoder neural network, a restricted Boltzmann machine, a support vector machine, a Bayesian classifier, K-means clustering, K Nearest Neighbors (KNN), transformer, recommender, and so on. When the ML model is a deep neural network, the model is trained using training data that includes features derived from data and labels corresponding to the data. For example, the data may be images of ECGs with a feature being the image itself or derived from the image (e.g., QRS integral), and the labels may be data indicated by the ECGs such as APLs or pacemaker configurations. The training results in a set of weights for the activation functions of the layers of the deep neural network. The trained deep neural network can then be applied to new data to generate a label for that new data. When the ML model is a support vector machine, a hyper-surface is found to divide the space of possible inputs. For example, the hyper-surface attempts to divide the pacemaker configurations associated APLs by maximizing the distance between the pacemaker configurations associated with an APL and the pacemaker configurations associated with other APLs to define the hyper-surface. The trained support vector machine can then be applied to new data to generate APL for a given pacemaker configuration. An ML model may generate values of discrete domain (e.g., classification), probabilities, and/or values of a continuous domain (e.g., regression value, classification probability).

An NN model has three major components: architecture, loss function, and search algorithm. The architecture defines the functional form relating the inputs to the outputs (in terms of network topology, unit connectivity, and activation functions). The search in weight space for a set of weights that minimizes the loss function is the training process. An NN model may use a radial basis function (RBF) network and a standard or stochastic gradient descent as the search technique with backpropagation.

An NN has multiple layers such as a convolutional layer a convolutional neural network (CNN), a rectified linear unit (ReLU) layer, a pooling layer, a fully connected (FC) layer, and so on. Some more complex NNs may have multiple convolutional layers, pooling layers, and FC layers. Each layer includes multiple neurons. A neuron inputs one or more outputs of a prior layer (or original input) and applies an activation function to the inputs to generate an output. For example, an NN may have multiple ReLU layers that each comprises multiple neurons (e.g., 10 layers with 128 neurons per layer) that each has an ReLU as an activation function. For example, the architecture of the APL evaluation ML model may have an input layer with neuron that each inputs multiple features of a pacemaker configuration. For example, a neuron may input each lead position, and other neurons may input different configuration parameters such as voltage and current.

A convolutional layer may include multiple filters (also referred to as kernels or activation functions). A filter inputs a convolutional window, for example, of a CT slice of a heart image, applies weights to each pixel of the convolutional window, and outputs a value for that convolutional window. For example, if the static image is 256 by 256 pixels, the convolutional window may be 8 by 8 pixels. The filter may apply a different weight to each of the 64 pixels in a convolutional window to generate the value. The convolutional layer may include, for each filter, a node (also referred to as a neuron) for each pixel of the CT slice assuming a stride of one with appropriate padding. Each node outputs a feature value based on a set of weights for the filter that are learned. The PCI system may employ a CNN when segmenting a CT scan of a patient or when identifying an APL based on an ECG image.

An activation function has a weight for each input and generates an output by combining the inputs based on the weights. The activation function may be a ReLU that sums the values of each input times its weight to generate a weighted value and outputs max(0,weighted value) to ensure that the output is not negative. The weights of the activation functions are learned when training a ML model. The ReLU function of max(0,weighted value) may be represented as a separate ReLU layer with a neuron for each output of the prior layer that inputs that output and applies the ReLU function to generate a corresponding “rectified output.”

A pooling layer may be used to reduce the size of the outputs of the prior layer by downsampling the outputs. For example, each neuron of a pooling layer may input 16 outputs of the prior layer and generate one output resulting in a 16-to-1 reduction in outputs.

An FC layer includes neurons that each input all the outputs of the prior layer and generate a weighted combination of those inputs. For example, if the penultimate layer generates 256 outputs and the FC layer inputs a neuron for each of three classifications, each neuron inputs the 256 outputs and applies weights to generate value for its classification.

Multimodal ML combines different modalities of input data to make a prediction. The modalities may be, for example, images, text, and ECGs.

In one multimodal ML approach, referred to as “early fusion,” data of the different modalities is combined at the input stage and is then trained on the multimodal data. The training data for these modalities include a collection of sets of an image, related text (e.g., derived from an EHR), and related ECGs and labels. The image, text, and ECGs may be used in their original form or preprocessed, for example, to reduce its dimensionality by compressing the data into byte arrays or applying a principal component analysis. A byte array may be processed by a cross-attention mechanism to condense the bytes into a vector of a fixed size. The vectors are then used to train an ML model which may be a supervised, self-supervised, or unsupervised ML model.

In a second multimodal ML approach, data from different modalities may be kept separate at the input stage and used as inputs to different, modality-specific ML models (e.g., a CNN for image data, a transformer for text, and a recurrent neural network (RNN) for a voltage-time series representation of an ECG). The modality-specific ML models may be trained jointly such that information from across different modalities is combined to make predictions, and the combined (cross-modality) loss is used to adjust model weights. Alternatively, the modality-specific ML models may also be trained separately using a separate loss function for each modality. A combined ML model is then trained based on the outputs of the modality specific models. Continuing with the example, the training data for each modality-specific ML model may be based on its data along with a label. The combined ML model is then trained with the outputs of the modality-specific ML models with a final label.

Transformer machine learning was introduced as an alternative to an RRN that is both more effective and more parallelizable. (See, Vaswani, A., Shazeer, N., Parmar, N., Uszkoreit, J., Jones, L., Gomez, A. N., Kaiser, Ł. and Polosukhin, I., 2017. Attention is all you need. Advances in neural information processing systems, 30, which is hereby incorporated by reference.) Transformer machine learning was originally described in the context of natural language processing (NLP) but has been adapted to other applications such as image processing to augment or replace a CNN. In the following, transformer machine learning is described in the context of NLP as introduced by Vaswani.

A transformer ML model includes an encoder whose output is input to a decoder. The encoder includes an input embedding layer followed by one or more encoder attention layers. The input embedding layer generates an embedding of the inputs. For example, if a transformer ML model is used to process a sentence as described by Vaswani, each word may be represented as a token that includes an embedding of a word and its positional information. The embedding of the word is a vector representation of the word. The embeddings of the words have the characteristic that words with similar meanings are closer in the vector space. The positional information is based on the position of the word in the sentence.

The first encoder attention layer inputs the embeddings and the other encoder attention layers input the output from the prior encoder attention layer. An encoder attention layer includes a multi-head attention mechanism followed by a normalization sublayer whose output is input to a feedforward NN followed by a normalization sublayer. A multi-head attention mechanism includes multiple self-attention mechanisms that each inputs the encodings of the previous layer and weighs the relevance of each encoding to other encodings. For example, the relevance may be determined by the following attention function:

$$Attention ( Q , K , V ) = softmax ( \frac{{QK}^{T}}{\sqrt{{d}_{k}}} ) V$$

where Q represents a query, K represents a key, V represents a value, and dk represents the dimensionality of K. This attention function is referred to as scaled dot-product attention. In Vaswani, the query, key, and value of an encoder multi-head attention mechanism is set to the input of the encoder attention layer. The multi-head attention mechanism determines the multi-head attention as represented by the following:

$$MultiHead ( Q , K , V ) = concat ( {head}_{1} , … , {head}_{8} ) {W}^{o}$$

$${head}_{i} = Attention ( {QW}_{i}^{Q} , {KW}_{i}^{K} , {VW}_{i}^{V} )$$

where W represents weights that are learned during training. The weights for the feedforward networks are also learned during training. The weights may be initialized to random values. A normalization layer normalizes its input to a vector having a dimension as expected by the next layer or sub-layer.

The decoder includes an output embedding layer, decoder attention layers, a linear layer, and a softmax layer. The output embedding layer inputs the output of the decoder shifted right. Each decoder attention layer inputs the output of the prior decoder attention layer (or the output embedding layer) and the output of the encoder. The embedding layer is input to the decoder attention layer, the output of the decoder attention layer is input the linear layer, and the output of the linear layer is input to the softmax layer which outputs probabilities. A decoder attention layer includes a decoder masked multi-head attention mechanism followed by a normalization sublayer, a decoder multi-head attention mechanism followed by a normalization sublayer, and a feedforward neural network followed by a normalization sublayer. The decoder masked multi-head attention mechanism masks the input so that predictions for a position are only based on outputs for prior positions. A decoder multi-head attention mechanism inputs the normalized output of the decoder masked multi-head attention mechanism as a query and the output of the encoder as a key and a value. The feedforward neural network inputs the normalized output of the decoder multi-head attention mechanism. The normalized output of the feedforward neural network is the output of that multi-head attention layer. The weights of the linear layer are also learned during training.

After being trained, a sentence may be input to the encoder to generate an encoding of the sentence that is input to the decoder. Initially, the output of the decoder that is input to the decoder is set to null. The decoder then generates an output based on the encoding and the null input. The output of the decoder is appended to the decoder's current input, and the decoder generates a new output. This decoding process is repeated until the encoder generates a termination symbol.

Although initially developed to process sentences as described above, transformers have been adapted for image classification. The input to a decoder of a transformer may be a representation of fixed-size patches of the image. (See, Dosovitskiy, A., Beyer, L., Kolesnikov, A., Weissenborn, D., Zhai, X., Unterthiner, T., Dehghani, M., Minderer, M., Heigold, G., Gelly, S. and Uszkoreit, J., 2020. An image is worth 16×16 words: Transformers for image recognition at scale. arXiv preprint arXiv:2010.11929, which is hereby incorporated by reference.) The representation of a patch may be, for each pixel of the patch, an encoding of its row, column, and color. The output of the encoder is fed into an NN to generate a classification of the image. The PCI system may employ a transformer to, for example, identify a pacemaker configuration or an APL given an image of an ECG and data derived from an EHR.

The PCI system may also employ a state space model (SSM) to generate a latent representation of cardiogram that is used for training. An example of an SSM is S4 as described in Gu, A. and Dao, T., 2023. Mamba: Linear-time sequence modeling with selective state spaces. arXiv preprint arXiv: 2312.00752 (Mamba), which is hereby incorporated by reference. Mamba provides a unique selection mechanism that adapts structured SSM parameters based on the input to selectively focus on relevant information within sequences, effectively filtering out less pertinent information. Mamba integrates SSM with multi-layer perceptron (MLP) blocks to support sequence modeling for sequential data such as cardiograms.

An unsupervised ML technique trains an ML model using unlabeled training data. K-means clustering is an example ML technique. Given feature vectors representing the training data, k-means clustering clusters the feature vectors into cluster of similar feature vectors. With k-means clustering, the number of clusters may be predefined. For example, the classification system may employ 50 clusters (k=50) to represent a cluster for each of 50 APL. An example training technique initially randomly places a feature vector (e.g., pacemaker configuration data) in each cluster. The training then repeatedly calculates a mean feature vector of each cluster, selects a feature vector not in a cluster, identifies the cluster whose mean is most similar, adds the feature vector to that cluster, and moves the feature vectors already in the clusters to the cluster with the most similar mean. Similarity may be determined, for example, based on Pearson similarity, cosine similarity, and so on. The training ends when all the feature vectors have been added to a cluster. An APL is then assigned to each cluster, for example, manually or automatically based on known APLs associated with some of the pacemaker configurations in that cluster. To identify an APL for a pacemaker configuration, a feature vector is generated based on the pacemaker configuration, the cluster with a mean that is most similar to that feature vector is identified, and the APL that is assigned is the APL for that pacemaker configuration.

Self-supervised learning is an ML technique that is based on unlabeled training data. Initially, self-supervised learning augments the training data to generate additional training data to generate sets of training data that are similar. For example, if the training data is ECG images, the self-supervised learning generates, for each ECG image, ECG images of varied timings and voltages. An ML model may have an encoder layer, pretext task layer, and contrastive learning component. The encoder layer encodes the images into a latent vector. The pretext task layer includes weights for grouping images into different clusters based on their differences using contrastive learning. Contrastive learning component employs a loss function for contrasting the images and adjusting the weights of the encoder and the pretext task layer. This approach is similar to k-means clustering but based on contrast rather than similarity. The weights of the pretext layer can be used as initial weights of a primary task such as training an suggest APL ML model which can be trained using labeled training data. Self-supervised learning may be performed on multimodal data as described above.

An ML decision tree defines information (e.g., APL) that is associated with entities (e.g., patients) that have certain characteristics. An ML decision may have the same form as a manually generated decision tree. However, the feature associated with each decision node of an ML decision tree may be selected automatically based on analysis of the training data. Each decision node (i.e., non-leaf node) of a decision tree corresponds to a feature and each branch from a decision node may correspond to a value or range of values for the feature of that decision node. For example, a decision node corresponding to a cardiac characteristic that is heart wall thickness may have branches for thin, normal, thick, and very thick, a decision node corresponding to a cardiac characteristic that is heart wall strain may have branches for normal, high, and very high, a decision node corresponding to AHA segments may have a branch for each AHA segment, and a decision node corresponding to lead configuration may have branches for a lead with four electrodes and a lead with eight electrodes. The leaf nodes of the decision tree may indicate an APL. The APL of a leaf node may be appropriate for a patient with features (e.g., QRS integral and wall thickness) that match the values of the features along the path from the root node to that leaf node.

For an ML decision tree, an entropy score may be used by an ML decision tree generator to select the feature to be associated with each decision node. The features selected depend on the patient data that is available. For example, one medical center may have EHRs with a field for wall thickness (e.g., determined by a heath provider) and another medical center may not. In such as case, different decision trees would be needed for the medical centers if one uses wall thickness data to identify an APL. The entropy score, for a possible feature for a decision node, is based on the distribution of its values in node feature vectors for that node. A node feature vector for a decision node has the values of the branches along the path from the root node to that decision node. If a first possible feature for a decision node has an equal number of node feature vectors for each value, the entropy for the first possible feature is considered to be high. In contrast, if a second possible feature has node feature vectors for which 75% have the same value, then the entropy for the second possible feature is considered to be low. In such a case, the ML decision tree generator would select the second possible feature for that decision node. The ML decision tree generator may also analyze features with continuous values to identify cut points that tend to minimize entropy. For example, as mentioned above, heart wall thickness may be categorized as normal, thick, and very thick. However, since the heart wall thickness of an EHR may be a continuous value, the PCI system may employ techniques to identify cut points (e.g., 4 or 5) that would reduce the entropy rather than the cut points for normal, thick, and very thick. Techniques for identifying cut points are described in Fayyad, U. M. and Irani, K. B., 1992. On the handling in decision tree of continuous-valued attributes generation. Machine Learning, 8, pp. 87-102, which is hereby incorporated by reference.

An ML decision tree generator may employ a depth-first, recursive algorithm to build the ML decision tree. The ML decision tree generator may employ a path termination criterion to determine when to terminate a path. The path termination criterion may be, for example, when the percentage of node feature vectors that are associated with the same wall thickness is above a threshold percentage. For example, if 95% of the node feature vectors have the same wall thickness, the ML decision tree generator may add a leaf node that indicates that 95% of cohort patients had a successful pacemaker implant used a certain pacemaker configuration or one similar to it. Other termination criteria may be that the number of node feature vectors is below a threshold number, or the path has reached a maximum depth. In such cases, the ML decision tree generator may add a leaf node that indicates that a pacemaker configuration recommendation cannot be provided or that the pacemaker configuration recommendation has a low confidence.

To provide recommendation for a patient, a feature vector is generated for the patient and the values of the features of the patient feature vector are used to identify the path that the patient feature vector matches. The pacemaker configuration recommendation is based on the leaf node of that path.

In some embodiments, a KNN model provides information relating to a patient. The training data for a KNN model may be training feature vectors (e.g., based on cardiac characteristics) and a label for each feature vector indicating a pacemaker configuration. A KNN model may be used without a training phase that is without learning weights or other parameters to represent the training data. In such a case, the patient feature vector is compared to the training feature vectors to identify a number (e.g., represented by the “K” in KNN) of similar training feature vectors. Once the number of similar training feature vectors are identified, the labels associated with the similar training feature vectors are analyzed to provide information for the patient. The labels of the training feature vectors that are more similar to a patient feature vector may be given a higher weight than those that are less similar. For example, if k is 10 and four training feature vectors are very similar and six are less similar, similarity weights of 0.9 may be assigned to the very similar training feature vectors and 0.2 to the less similar. If three of the four and one of the six have the same information, then the information for the patient is primarily based on that information even though most of the 10 have different information. Conceptually, training feature vectors that are very similar are closer to the entity feature vector in a multi-dimensional space of features and a similarity weight is based on distance between the feature vectors. Various techniques may be employed to calculate a similarity metric indicating similarity between a patient feature vector and a training feature vector such as a dot product, cosine similarity, Pearson correlation, and so on.

If the number of training feature vectors is large, various techniques may be employed to effectively “compress” the training data during a training phase. For example, a clustering technique may be employed to identify clusters of training feature vectors that are similar and have the same label. A training feature vector may be generated for each cluster (e.g., one from the cluster or one based on mean values for the features) as a cluster feature vector and assign a cluster weight to it based on number of training feature vectors in the cluster.

The PCI system may employ a recommender ML system to identify a pacemaker configuration based on an APL and/or cardiac characteristics, based on pacemaker configurations of clinic data or simulated data that are deemed successful based on APLs and cardiac characteristics. A recommender ML system may analyze large volumes of clinical data to identify patterns aiming to curate personalized pacemaker configuration recommendations. By leveraging clinical data of successful pacemaker implantations, the recommender ML system can offer suggestions that are tailored to data derived from a patient's EHR and data provided by a health care provider. A recommender ML system may employ various ML algorithms such as collaborative filtering. Collaborative filtering focuses on finding similarities among the EHR of patients who have had successful pacemaker implantations. By identifying patterns of similarity, the recommender ML system can predict how successful a pacemaker configuration would be for a patient. The recommender ML system may employ matrix factorization to reduce a large patient-pacemaker configuration matrix into lower-dimensional matrices, identifying latent characteristics of patients and pacemaker configurations. NNs and deep learning approaches may be employed to further refine recommendations by capturing non-linear relationships and complex patterns within the data, accommodating a richer set of features such as images and the evolution of a medical condition.

In some embodiments, the number of training examples (e.g., feature vectors and labels) of a training dataset based on clinical and/or simulated may not be sufficient to effectively train an ML model. To overcome this insufficiency, the PCI system may employ a diffusion ML model to generate additional training data using a generative process. (See, Rombach, R., Blattmann, A., Lorenz, D., Esser, P. and Ommer, B., 2022. High-resolution image synthesis with latent diffusion models. In Proceedings of the IEEE/CVF conference on computer vision and pattern recognition (pp. 10684-10695), which is hereby incorporated by reference.) A diffusion ML model is a generative ML model that inputs noisy data and progressively denoises the data until the denoised data appears to be indistinguishable from real data such as an image of an ECG or a 3D mesh having values associated with vertices. A diffusion ML model is trained using a forward diffusion process that successively adds noise to input training data such as ECG images to generate noisy data and a reverse diffusion process that successively denoises the noisy data to generate denoised data that approximates the input training data. The training learns weights for the reverse diffusion process that tend to minimize the difference between the input training data and the denoised data. After a diffusion model is trained, the reverse diffusion process is employed to generate data that can be used to train an ML model such as an evaluate APL ML model. To do so, randomly generated noisy data is input to the reverse diffusion process which denoises the noisy data to generate the denoised data that appears to real data.

The forward diffusion process employs a Markov chain that incrementally adds Gaussian noise to the training data over a series of steps. This process transforms the training data from its initial distribution to a Gaussian distribution. The reverse diffusion process employs a neural network to incrementally approximate and remove the noise that was added at each step of the forward diffusion process. When generating data, randomly generated noisy data is input to the reverse diffusion process which incrementally removes the noise that was learned during training.

The forward diffusion process systematically adds Gaussian noise to the original data x₀ Gaussian noise over T timesteps, resulting in a sequence of increasingly noisy data x₁, x₂, . . . , x_{T}. The process at each time step t may be represented by the equation:

$${x}_{t} = \sqrt{{α}_{t}} {x}_{t - 1} + \sqrt{1 - {α}_{t}} {ε}_{t} , {ε}_{t} \sim 𝒩 ( 0 , I )$$

where xₜ is data at timestep t, εₜ is Gaussian noise, αₜ is the amount of noise added, and I is the identity matrix.

The reverse diffusion process learns the distribution of the training data by starting from noise and progressively denoising it over the timesteps. The training estimates the reverse of the forward diffusion process using a neural network that may be represented by the equation:

$${x}_{t - 1} = \frac{1}{\sqrt{{α}_{t}}} ( {x}_{t} - \frac{1 - {α}_{t}}{\sqrt{1 - {\overline{α}}_{t}}} {f}_{θ} ( {x}_{t} , t ) )$$

where

$${\overline{α}}_{t} = {\prod }_{i = 1}^{t} {α}_{i}$$

represent the cumulative noise and f_{θ}(xₜ,t) represents the neural network.

The goal of training a diffusion model is to minimize the difference between the original data and the data reconstructed by the reverse diffusion process using a loss function that may be represented by the equation.

$$L ( θ ) = {𝔼}_{{x}_{0} , {ε}_{t} , t} [ {‖ {ε}_{t} - {f}_{θ} ( {x}_{t} , t ) ‖}^{2} ] .$$

A diffusion model may also include a conditioning mechanism that allows for factoring in a domain-specific information into the reverse diffusion process. The domain-specific information may be employed by a cross-attention mechanism of the neural network (e.g., U-Net architecture) of the reverse diffusion process.

In some embodiment, the PCI system may employ a generative adversarial network (GAN) to, for example, generate additional training examples or implement one of the ML models of the PCI system. (See, Goodfellow, I., Pouget-Abadie, J., Mirza, M., Xu, B., Warde-Farley, D., Ozair, S., Courville, A. and Bengio, Y., 2020. Generative adversarial networks. Communications of the ACM, 63(11), pp. 139-144, which is hereby incorporated by reference). A GAN employs a generator and discriminator and is trained using training data such as real cardiac characteristics. The generator generates cardiac characteristics based on random input. The generator is trained to generate data that cannot be distinguished from real data such as cardiac characteristics. The discriminator indicates whether input cardiac characteristics are real or generated. The generator and discriminator are trained in parallel to learn weights. The generator is trained to generate increasingly more realistic cardiac characteristics, and the discriminator is trained to discriminate between real cardiac characteristics and generated cardiac characteristics more effectively. After being trained, the generator can be used to generate cardiac characteristics that are realistic, and the discriminator can be used to discriminate between real cardiac characteristics and generated (fake) cardiac characteristics. However, the discriminator may not be employed to perform such discrimination.

The following paragraphs describe various aspects of the PCI system. An implementation of the PCI system may employ any combination or sub-combination of the aspects and may employ additional aspects. The processing of the aspects may be performed by one or more computing systems with one or more processors that execute computer-executable instructions that implement the aspects and that are stored on one or more computer-readable storage mediums.

In some aspects, the techniques described herein relate to a method performed by one or more computing systems, the method including: accessing a pacemaker configuration; identifying an apparent activation location for the pacemaker configuration; generating a graphic of a heart; adding an indication of the apparent activation location to the graphic; and displaying the graphic of the heart with the indication of the apparent activation location. In some aspects, the techniques described herein relate to a method wherein the identifying of the apparent activation location includes: accessing a library that includes associations between library pacemaker configurations and library apparent activation locations; identifying a library pacemaker configuration of the library that is similar to the pacemaker configuration wherein similarity is based on a pacemaker similarity criterion wherein the identified apparent activation location is the library apparent activation location associated with the identified library pacemaker configuration. In some aspects, the techniques described herein relate to a method wherein the identifying of the apparent activation location includes inputting the pacemaker configuration to a machine learning model that outputs the apparent activation location. In some aspects, the techniques described herein relate to a method further including for each of a plurality of cardiac characteristics and pacemaker configurations, running a simulation of electrical activity of a heart having those cardiac characteristics based on pacing the heart with a pacemaker having that pacemaker configuration, determining an apparent activation location based on the simulated electrical activity, and associating that pacemaker configuration with that apparent activation location. In some aspects, the techniques described herein relate to a method further including adding to the graphic an indication of one or more nonresponsive segment. In some aspects, the techniques described herein relate to a method further including adding to the graphic indications of locations of electrodes specified by the pacemaker configuration. In some aspects, the techniques described herein relate to a method further including generating an effectiveness evaluation for the apparent activation location by inputting the apparent activation location to a machine learning model that outputs an effectiveness score. In some aspects, the techniques described herein relate to a method wherein after the graphic is displayed, a pacemaker implantation procedure is performed on a patient to implant a pacemaker having the pacemaker configuration. In some aspects, the techniques described herein relate to a method further including: accessing cardiac characteristics of a patient; and inputting the cardiac characteristics to a machine learning model that outputs the pacemaker configuration.

In some aspects, the techniques described herein relate to a method performed by one or more computing systems, the method including: for each of a plurality of cardiac characteristics and pacemaker configurations, running a simulation of electrical activity of a heart having those cardiac characteristics based on pacing the heart with a pacemaker having that pacemaker configuration; determining an apparent activation location for that pacemaker configuration based on the simulated electrical activity; and associating that pacemaker configuration with that apparent activation location and the cardiac characteristics. In some aspects, the techniques described herein relate to a method wherein for each simulation of electrical activity, the determining of the apparent activation location for a pacemaker configuration includes generating a simulated cardiogram based on the simulated electrical activity and inputting the simulated cardiogram to a mapping system that outputs the apparent activation location. In some aspects, the techniques described herein relate to a method wherein a pacemaker configuration includes a specification of one or more leads and one or more electrodes per lead and a specification of parameter setting. In some aspects, the techniques described herein relate to a method further including training a machine learning model that inputs cardiac characteristics and/or a cardiogram and outputs a pacemaker configuration, the machine learning model being trained with a training dataset of training examples, each training example having a feature vector derived from cardiac characteristics and/or a cardiogram of a simulation and having a label based on the pacemaker configuration of that simulation. In some aspects, the techniques described herein relate to a method further including accessing cardiac characteristics of a patient and inputting those cardiac characteristics to the machine learning model that outputs a pacemaker configuration. In some aspects, the techniques described herein relate to a method wherein a pacemaker implantation procedure is performed on a patient to implant a pacemaker having the output pacemaker configuration. In some aspects, the techniques described herein relate to a method further including training a machine learning model that inputs cardiac characteristics and/or a cardiogram and outputs an apparent activation location, the machine learning model being trained with a training dataset of training examples, each training example having a feature vector derived from cardiac characteristics and/or a cardiogram of a simulation and having a label based on the apparent activation location of that simulation. In some aspects, the techniques described herein relate to a method further including accessing cardiac characteristics and/or a cardiogram of a patient and inputting those cardiac characteristics and/or cardiogram to the machine learning model that outputs an apparent activation location. In some aspects, the techniques described herein relate to a method wherein a pacemaker implantation procedure is performed on a patient to implant a pacemaker with a pacemaker configuration that would result in the output apparent activation location. In some aspects, the techniques described herein relate to a method further including training a machine learning model that inputs an apparent activation location and cardiac characteristics and/or a cardiogram and outputs pacemaker configuration, the machine learning model being trained with a training dataset of training examples, each training example having a feature vector derived from an apparent activation location and cardiac characteristics and/or a cardiogram of a simulation and having a label based on the pacemaker configuration of that simulation. In some aspects, the techniques described herein relate to a method further including accessing a desired apparent activation location for and cardiac characteristics and/or a cardiogram of a patient and inputting the desired apparent activation location and cardiac characteristics and/or cardiogram to the machine learning model that outputs a pacemaker configuration. In some aspects, the techniques described herein relate to a method wherein a pacemaker implantation procedure is performed on a patient to implant a pacemaker configures based on the output pacemaker configuration. In some aspects, the techniques described herein relate to a method further including training a machine learning model that inputs a pacemaker configuration and cardiac characteristics and/or a cardiogram and outputs an apparent activation location, the machine learning model being trained with a training dataset of training examples, each training example having a feature vector derived from a pacemaker configuration and cardiac characteristics and/or a cardiogram of a simulation and having a label based on the apparent activation location of that simulation. In some aspects, the techniques described herein relate to a method further including accessing a pacemaker configuration and cardiac characteristics and/or a cardiogram of a patient and inputting the pacemaker configuration and cardiac characteristics and/or cardiogram to the machine learning model that outputs an apparent activation location. In some aspects, the techniques described herein relate to a method wherein a pacemaker implantation procedure is performed on a patient to implant a pacemaker configures based on the output pacemaker configuration. In some aspects, the techniques described herein relate to a method further including applying a non-machine learning algorithm to determine a pacemaker configuration and/or an apparent activation location for a patient having a patient cardiac characteristics and desired patient pacemaker configuration and/or a desired apparent activation location based on the associations of pacemaker configurations with apparent activation locations and cardiac characteristics. In some aspects, the techniques described herein relate to a method wherein a simulation is run until the simulated electrical activity stabilizes. In some aspects, the techniques described herein relate to a method wherein when the electrical activity of a simulation stabilizes into a normal sinus rhythm, indicating the pacemaker configuration is effective for treating a patient with the cardiac characteristics of that simulation.

In some aspects, the techniques described herein relate to a method performed by one or more computing systems, the method including: for each of a plurality of electronic health records of patients who have had pacemaker implantations, accessing electronic health record data that includes one or more of a pacemaker configuration, cardiac characteristics of that patient, and a pre-implantation arrhythmia cardiogram collected from the patient; accessing a post-implantation cardiogram collected from the patient; inputting to the post-implantation cardiogram to a mapping system that outputs an apparent activation location; and associating the electronic health record data with the apparent activation location. In some aspects, the techniques described herein relate to a method further including training a machine learning model that inputs cardiac characteristics and/or a pre-implantation cardiogram and outputs a pacemaker configuration, the machine learning model being trained with a training dataset of training examples, each training example derived from an electronic health record and having a feature vector derived from cardiac characteristics and/or a pre-implantation cardiogram of that electronic health record and having a label based on the pacemaker configuration of that electronic health record. In some aspects, the techniques described herein relate to a method further including training a machine learning model that inputs electronic health record data and outputs an apparent activation location, the machine learning model being trained with a training dataset of training examples, each training example derived from an electronic health record having a feature vector derived from the electronic health record data of that electronic health record and having a label based on the apparent activation location derived from a post-implantation cardiogram of the electronic health record. In some aspects, the techniques described herein relate to a method including training a machine learning model that inputs electronic health record data and outputs an apparent activation location, the machine learning model being trained with a training dataset of training examples, each training example derived from an electronic health record having a feature vector derived from the electronic health record data of that electronic health record and having a label based on the apparent activation location derived from a post-implantation cardiogram of the electronic health record. In some aspects, the techniques described herein relate to a method including training a machine learning model that inputs an apparent activation location and cardiac characteristics and/or a pre-implantation cardiogram and outputs an indication of effectiveness of that apparent activation location in treating a patient having those cardiac characteristics and/or that pre-implantation cardiogram, the machine learning model being trained with a training dataset of training examples, each training example derived from an electronic health record having a feature vector derived from the apparent activation location and the cardiac characteristics and/or a post-implantation cardiogram of that electronic health record and having a label indicating effectiveness of the apparent activation location. In some aspects, the techniques described herein relate to a method wherein the effectiveness of a label is determined based on analysis of the post-implantation cardiogram of that electronic health record. In some aspects, the techniques described herein relate to a method including training a machine learning model that inputs an cardiac characteristics and/or a pre-implantation cardiogram and outputs an indication a pacemaker configuration that may be effective in treating a patient having those cardiac characteristics and/or that pre-implantation cardiogram, the machine learning model being trained with a training dataset of training examples, each training example derived from an electronic health record having a feature vector derived from a cardiac characteristics and/or a post-implantation cardiogram of that electronic health record and having a label indicating a pacemaker configuration of that electronic health record.

In some aspects, the techniques described herein relate to a method for treating a patient with a cardiac resynchronization therapy, the method including: under control of one or more computing systems, receiving a pacemaker configuration of a pacemaker; determining an apparent activation location based on a computational model assuming implantation of the pacemaker having the pacemaker configuration; assessing whether implantation of the pacemaker having the pacemaker configuration would be effective at treating the patient; and outputting an indication of the assessment; and based on analysis of the assessment, implanting the pacemaker having the pacemaker configuration in the patient. In some aspects, the techniques described herein relate to a method further including under control of the one or more computing systems, generating a graphic of a heart that includes an indication of the apparent activation location and displaying the graphic. In some aspects, the techniques described herein relate to a method wherein the graphic included indications of nonresponsive segments.

In some aspects, the techniques described herein relate to a method for implanting a pacemaker to assist in synchronizing the rhythm of the heart a patient, the method including: during an implantation procedure, inputting to a pacemaker implantation system an indication of cardiac characteristics of the patient and/or a pre-implantation cardiogram collected from the patient; receiving from the pacemaker implantation system an indication of a pacemaker configuration and an indication of an apparent activation location; and implanting the pacemaker in patient based on the pacemaker configuration to help synchronize the rhythm the heart of the patient.

In some aspects, the techniques described herein relate to a method for implanting a pacemaker to assist in synchronizing the rhythm of the heart a patient, the method including: during an implantation procedure, inputting to a pacemaker configuration identification system an apparent activation location and an indication of cardiac characteristics of the patient and/or a pre-implantation cardiogram collected from the patient; receiving from the pacemaker configuration identification system an indication of a pacemaker configuration; and implanting the pacemaker in patient based on the pacemaker configuration to help synchronize the rhythm the heart of the patient.

In some aspects, the techniques described herein relate to one or more computing systems for providing information relating to an implantation of a pacemaker as a cardiac resynchronization therapy for a patient, the one or more computing systems including: one or more computer-readable storage mediums that store computer-executable instructions for controlling the one or more computing systems to: access a desired apparent activation location for the heart of the patient after implantation of the pacemaker; identify a pacemaker configuration that would likely result in the heart having the desired activation location; and output an indication of the pacemaker configuration; and one or more processors for controlling the one or more computing systems to execute one or more of the computer-executable instructions. In some aspects, the techniques described herein relate to one or more computing systems wherein the identified pacemaker configuration is a standardized pacing protocol.

In some aspects, the techniques described herein relate to a method including: under control of one or more computing systems, accessing cardiac data of a patient, the cardiac data including cardiac characteristics and/or a cardiogram; selecting a pacemaker configuration for a pacemaker; predicting, based on the cardiac data and the pacemaker configuration, an apparent activation location (APL) within a heart; generating a graphic representation of the heart with an indication of the predicted APL; and displaying the graphic representation during a pacemaker implantation procedure; and performing an implantation procedure based on the pacemaker configuration so that a cardiac cycle appears to be activated at the predicted APL during pacing by the pacemaker.

In some aspects, the techniques described herein relate to a system for assisting cardiac resynchronization therapy, including: one or more processors; one or more computer-readable media storing computer-executable instructions that, when executed, cause the processors to: access a pacemaker configuration; access patient cardiac data of a patient; predict an apparent activation location (APL) resulting from the pacemaker configuration factoring in the cardiac data; generate and display a graphic representation of the patient's heart with an indication of the APL; and provide recommendations for modifying the pacemaker configuration to optimize response to the cardiac resynchronization therapy.

All documents incorporated by reference are incorporated in their entirety for the full extent of their disclosures. In the event of inconsistencies between the language in this document and any incorporated-by-reference document, the language in the incorporated-by-reference document should be considered supplementary to that of this document and the language in this document controls.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

1. A method performed by one or more computing systems, the method comprising:
accessing a pacemaker configuration;
identifying an apparent activation location for the pacemaker configuration;
generating a graphic of a heart;
adding an indication of the apparent activation location to the graphic; and
displaying the graphic of the heart with the indication of the apparent activation location.

2. The method of claim 1 wherein the identifying of the apparent activation location includes:
accessing a library that includes associations between library pacemaker configurations and library apparent activation locations;
identifying a library pacemaker configuration of the library that is similar to the pacemaker configuration wherein similarity is based on a pacemaker similarity criterion
wherein the identified apparent activation location is the library apparent activation location associated with the identified library pacemaker configuration.

3. The method of claim 1 wherein the identifying of the apparent activation location includes inputting the pacemaker configuration to a machine learning model that outputs the apparent activation location.

4. The method of claim 1 further comprising for each of a plurality of cardiac characteristics and pacemaker configurations, running a simulation of electrical activity of a heart having those cardiac characteristics based on pacing the heart with a pacemaker having that pacemaker configuration, determining an apparent activation location based on the simulated electrical activity, and associating that pacemaker configuration with that apparent activation location.

5. The method of claim 1 further comprising adding to the graphic an indication of one or more nonresponsive segment.

6. The method of claim 1 further comprising adding to the graphic indications of locations of electrodes specified by the pacemaker configuration.

7. The method of claim 1 further comprising generating an effectiveness evaluation for the apparent activation location by inputting the apparent activation location to a machine learning model that outputs an effectiveness score.

8. (canceled)

9. The method of claim 1 further comprising:
accessing cardiac characteristics of a patient; and
inputting the cardiac characteristics to a machine learning model that outputs the pacemaker configuration.

10. A method performed by one or more computing systems, the method comprising:
for each of a plurality of cardiac characteristics and pacemaker configurations,
running a simulation of electrical activity of a heart having those cardiac characteristics based on pacing the heart with a pacemaker having that pacemaker configuration;
determining an apparent activation location for that pacemaker configuration based on the simulated electrical activity; and
associating that pacemaker configuration with that apparent activation location and the cardiac characteristics.

11. The method of claim 10 wherein for each simulation of electrical activity, the determining of the apparent activation location for a pacemaker configuration includes generating a simulated cardiogram based on the simulated electrical activity and inputting the simulated cardiogram to a mapping system that outputs the apparent activation location.

12. The method of claim 10 wherein a pacemaker configuration includes a specification of one or more leads and one or more electrodes per lead and a specification of parameter setting.

13. The method of claim 10 further comprising training a machine learning model that inputs cardiac characteristics and/or a cardiogram and outputs a pacemaker configuration, the machine learning model being trained with a training dataset of training examples, each training example having a feature vector derived from cardiac characteristics and/or a cardiogram of a simulation and having a label based on the pacemaker configuration of that simulation.

14. The method of claim 13 further comprising accessing cardiac characteristics of a patient and inputting those cardiac characteristics to the machine learning model that outputs a pacemaker configuration.

15. (canceled)

16. The method of claim 10 further comprising training a machine learning model that inputs cardiac characteristics and/or a cardiogram and outputs an apparent activation location, the machine learning model being trained with a training dataset of training examples, each training example having a feature vector derived from cardiac characteristics and/or a cardiogram of a simulation and having a label based on the apparent activation location of that simulation.

17. The method of claim 16 further comprising accessing cardiac characteristics and/or a cardiogram of a patient and inputting those cardiac characteristics and/or cardiogram to the machine learning model that outputs an apparent activation location.

18. (canceled)

19. The method of claim 10 further comprising training a machine learning model that inputs an apparent activation location and cardiac characteristics and/or a cardiogram and outputs pacemaker configuration, the machine learning model being trained with a training dataset of training examples, each training example having a feature vector derived from an apparent activation location and cardiac characteristics and/or a cardiogram of a simulation and having a label based on the pacemaker configuration of that simulation.

20. The method of claim 19 further comprising accessing a desired apparent activation location for and cardiac characteristics and/or a cardiogram of a patient and inputting the desired apparent activation location and cardiac characteristics and/or cardiogram to the machine learning model that outputs a pacemaker configuration.

21. (canceled)

22. The method of claim 10 further comprising training a machine learning model that inputs a pacemaker configuration and cardiac characteristics and/or a cardiogram and outputs an apparent activation location, the machine learning model being trained with a training dataset of training examples, each training example having a feature vector derived from a pacemaker configuration and cardiac characteristics and/or a cardiogram of a simulation and having a label based on the apparent activation location of that simulation.

23. The method of claim 22 further comprising accessing a pacemaker configuration and cardiac characteristics and/or a cardiogram of a patient and inputting the pacemaker configuration and cardiac characteristics and/or cardiogram to the machine learning model that outputs an apparent activation location.

24. (canceled)

25. The method of claim 10 further comprising applying a non-machine learning algorithm to determine a pacemaker configuration and/or an apparent activation location for a patient having a patient cardiac characteristics and desired patient pacemaker configuration and/or a desired apparent activation location based on the associations of pacemaker configurations with apparent activation locations and cardiac characteristics.

26. The method of claim 10 wherein a simulation is run until the simulated electrical activity stabilizes.

27. The method of claim 26 wherein when the electrical activity of a simulation stabilizes into a normal sinus rhythm, indicating the pacemaker configuration is effective for treating a patient with the cardiac characteristics of that simulation.

28. A method performed by one or more computing systems, the method comprising:
for each of a plurality of electronic health records of patients who have had pacemaker implantations,
accessing electronic health record data that includes one or more of a pacemaker configuration, cardiac characteristics of that patient, and a pre-implantation arrhythmia cardiogram collected from the patient;
accessing a post-implantation cardiogram collected from the patient;
inputting to the post-implantation cardiogram to a mapping system that outputs an apparent activation location; and
associating the electronic health record data with the apparent activation location.

29. The method of claim 28 further comprising training a machine learning model that inputs cardiac characteristics and/or a pre-implantation cardiogram and outputs a pacemaker configuration, the machine learning model being trained with a training dataset of training examples, each training example derived from an electronic health record and having a feature vector derived from cardiac characteristics and/or a pre-implantation cardiogram of that electronic health record and having a label based on the pacemaker configuration of that electronic health record.

30. The method of claim 28 further comprising training a machine learning model that inputs electronic health record data and outputs an apparent activation location, the machine learning model being trained with a training dataset of training examples, each training example derived from an electronic health record having a feature vector derived from the electronic health record data of that electronic health record and having a label based on the apparent activation location derived from a post-implantation cardiogram of the electronic health record.

31. The method of claim 28 comprising training a machine learning model that inputs electronic health record data and outputs an apparent activation location, the machine learning model being trained with a training dataset of training examples, each training example derived from an electronic health record having a feature vector derived from the electronic health record data of that electronic health record and having a label based on the apparent activation location derived from a post-implantation cardiogram of the electronic health record.

32. The method of claim 28 comprising training a machine learning model that inputs an apparent activation location and cardiac characteristics and/or a pre-implantation cardiogram and outputs an indication of effectiveness of that apparent activation location in treating a patient having those cardiac characteristics and/or that pre-implantation cardiogram, the machine learning model being trained with a training dataset of training examples, each training example derived from an electronic health record having a feature vector derived from the apparent activation location and the cardiac characteristics and/or a post-implantation cardiogram of that electronic health record and having a label indicating effectiveness of the apparent activation location.

33. The method of claim 32 wherein the effectiveness of a label is determined based on analysis of the post-implantation cardiogram of that electronic health record.

34. The method of claim 28 comprising training a machine learning model that inputs an cardiac characteristics and/or a pre-implantation cardiogram and outputs an indication a pacemaker configuration that may be effective in treating a patient having those cardiac characteristics and/or that pre-implantation cardiogram, the machine learning model being trained with a training dataset of training examples, each training example derived from an electronic health record having a feature vector derived from a cardiac characteristics and/or a post-implantation cardiogram of that electronic health record and having a label indicating a pacemaker configuration of that electronic health record.

35-39. (canceled)

40. One or more computing systems for providing information relating to an implantation of a pacemaker as a cardiac resynchronization therapy for a patient, the one or more computing systems comprising:
one or more computer-readable storage mediums that store computer-executable instructions for controlling the one or more computing systems to:
access a desired apparent activation location for the heart of the patient after implantation of the pacemaker;
identify a pacemaker configuration that would likely result in the heart having the desired activation location; and
output an indication of the pacemaker configuration; and
one or more processors for controlling the one or more computing systems to execute one or more of the computer-executable instructions.

41. The one or more computing systems of claim 40 wherein the identified pacemaker configuration is a standardized pacing protocol.

42. (canceled)

43. A system for assisting cardiac resynchronization therapy, comprising:
one or more processors;
one or more computer-readable media storing computer-executable instructions that, when executed, cause the processors to:
access a pacemaker configuration;
access patient cardiac data of a patient;
predict an apparent activation location (APL) resulting from the pacemaker configuration factoring in the cardiac data;
generate and display a graphic representation of the patient's heart with an indication of the APL; and
provide recommendations for modifying the pacemaker configuration to optimize response to the cardiac resynchronization therapy.
